(19) United States
(12) Patent Application Publication
(10) Pub. No.: US 2004/0058901 A1
(43) Pub. Date: Mar. 25, 2004

(54) Crystalline ethanolate solvate form of zafirluskast process for manufacture and pharmaceutical compositions thereof

(76) Inventors: Arie Gutman, Haifa (IL); Genadi Nisnevich, Haifa (IL); Igor Zaltzman, Haifa (IL); Boris Pertsikov, Haifa (IL); Maxim Sotrihin, Haifa (IL); Victor Ponomarev, Haifa (IL)

Correspondence Address: BROWDY AND NEIMARK, P.L.L.C., 624 NINTH STREET, NW, SUITE 300, WASHINGTON, DC, 20001-5303, US

(21) Appl. No.: 10/399,978

(22) PCT Filed: Oct. 24, 2001
(86) PCT No.: PCT/IL01/00982

(30) Foreign Application Priority Data
Oct. 24, 2000 (IL) 139243

(51) Int. Cl. A61K 31/33

(52) U.S. Cl. 514/183

(57) ABSTRACT

A novel, crystalline ethanolate solvate form of zafirlukast is disclosed as well as methods for preparing it. Pharmaceutical compositions containing this novel form of zafirlukast are also disclosed. The compositions are useful in the treatment of diseases in which leukotrienes are implicated for example in the treatment of asthma.

### FIELD OF THE INVENTION

This invention relates to a new crystalline form of zafirlukast, processes for its manufacture, and pharmaceutical compositions containing the new form.

### BACKGROUND OF THE INVENTION

Zafirlukast, cyclopentyl 3-[2-methoxy-4-[(o-tolylsulfonyl)carbamoyl]benzyl]-1-methylindole-5-carbamate, having the formula:

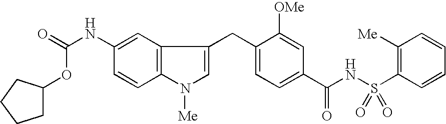

is a first anti-asthmatic leukotriene antagonist (Matassa, V. G. et al., J. Med. Chem., v. 33, 1781 (1990); U.S. Pat. No. 4,859,692 and The Merck Index, 12th Edition, 10241).

Zafirlukast may be prepared by the methods described in J. Med. Chem., v. 33, 1781 (1990) and Example 105 of U.S. Pat. No. 4,859,692. U.S. Pat. No. 5,993,859 discloses three physical forms of zafirlukast, designated Form A, Form B and Form X. These forms differ from one another in respect of their physical properties, stability, spectral data and methods of preparation. Form X is a physically stable crystalline form, but has relatively poor bioavailability. Form B is a physically unstable crystalline form of zafirlukast hydrate. The method disclosed in U.S. Pat. No. 5,993,859 for preparing the Form B is by precipitation with water from acetone. Because of its unstable nature, it is difficult to prepare this hydrate form. It is also difficult to maintain it in a form having a constant, reproducible water-content, and is particularly difficult to handle it during formulation. Form A is an amorphous form of zafirlukast prepared by dehydration of Form B in a vacuum oven for up to 24 hours at 120° C. Typically the pressure is about 20 mbar. Form A has relatively good bioavailability. However, it has been found that conversion of form A into form B occurs under conditions of high relative humidity and elevated temperatures.

Accordingly, it may in certain circumstances be desirable to keep pharmaceutical formulations comprising form A in the presence of a suitable desiccant, such as silica gel. It may also be desirable to keep them in an airtight container, such as a blister pack.

### SUMMARY OF INVENTION

The present invention provides a new solid ethanolate solvate form of zafirlukast (hereinafter the expression “ethanolate solvate form of zafirlukast” is interchangeable with “zafirlukast ethanolate”). Preferably, the solid form is crystalline. This new form is a relatively stable physical form of zafirlukast, and is less hygroscopic than Form A of zafirlukast.

The present invention also provides methods of preparing zafirlukast ethanolate.

The present invention further provides a pharmaceutical composition comprising an effective amount of zafirlukast ethanolate and pharmaceutically acceptable carriers.

The present invention also provides the use of zafirlukast ethanolate in the manufacture of a medicament. In addition, the present invention provides a method for the treatment of a disease state in which leukotrienes are implicated, such method comprising administering to a subject in need of such treatment, a therapeutically effective amount of zafirlukast ethanolate.

### BRIEF DESCRIPTION OF THE DRAWINGS

FIG. 1 shows schematically a perspective view of ethanolate solvate form of zafirlukast molecule and the atomic numbering of non-hydrogen atoms as derived from single crystal x-ray analysis. (Atomic coordinates based on Table 2).

FIG. 2 shows a characteristic powder x-ray diffraction pattern of zafirlukast ethanolate of the present invention. Vertical axis: intensity (CPS); Horizontal axis: 2θ (degrees).

FIG. 3 shows a plot of calculated versus experimental powder x-ray diffraction pattern of zafirlukast ethanolate. Vertical axis: intensity (CPS); Horizontal axis: 2θ (degrees).

FIG. 4 shows the infrared spectrum of zafirlukast ethanolate in potassium bromide.

FIG. 5 shows the ¹H nuclear magnetic resonance (NMR) spectrum of zafirlukast ethanolate.

FIG. 6 shows the differential scanning calorimetry (DSC) thermogram of zafirlukast ethanolate.

### DETAILED DESCRIPTION OF THE INVENTION

The present invention discloses, according to a first of its aspects, a new solid ethanolate solvate form of zafirlukast (zafirlukast ethanolate) that is less hygroscopic than other known forms of zafirlukast. Preferably, the solid solvate form is crystalline.

The crystalline state of a compound can be unambiguously described by several crystallographic parameters: unit cell dimensions, space group, and atomic position of all atoms in the compound relative to the origin of its unit cell. These parameters are experimentally determined by single crystal x-ray analysis. The crystalline zafirlukast ethanolate is characterized by the crystal parameters obtained from single crystal x-ray crystallographic analysis set forth in Table 1 below.

TABLE 1

| | Crystal parameters of |
|---|---|
| | zafirlukast ethanolate |
| Formula | C₃₁H₃₃N₃O₆S.C₂H₅OH |
| Formula weight, amu | 621.73 |
| Crystal size (mm) | 0.4 × 0.3 × 0.15 |
| Space group | Cc |
| Crystal system | monoclinic |
| Temperature, K | 293(2) |
| Cell dimensions | a = 17.2160(10) Å |
| (Å = Angstrom | b = 11.0330(10) Å |
| ° = degree) | c = 18.2580(10) Å |
| | α = 90.00° |
| | β = 109.467(10)° |
| | γ = 90.00° |
| V (Å³) | 3269.7 |
| Z (molecules/unit cell) | 4 |
| density (g/cm³) | 1.263 |

The unit cell dimension is defined by three parameters: length of the sides of the cell, relative angles of sides to each other and the volume of the cell. The lengths of the sides of the unit cell are defined by a, b and c. The relative angles of the cell sides are defined by α, β and γ. The volume of the cell is defined as V.

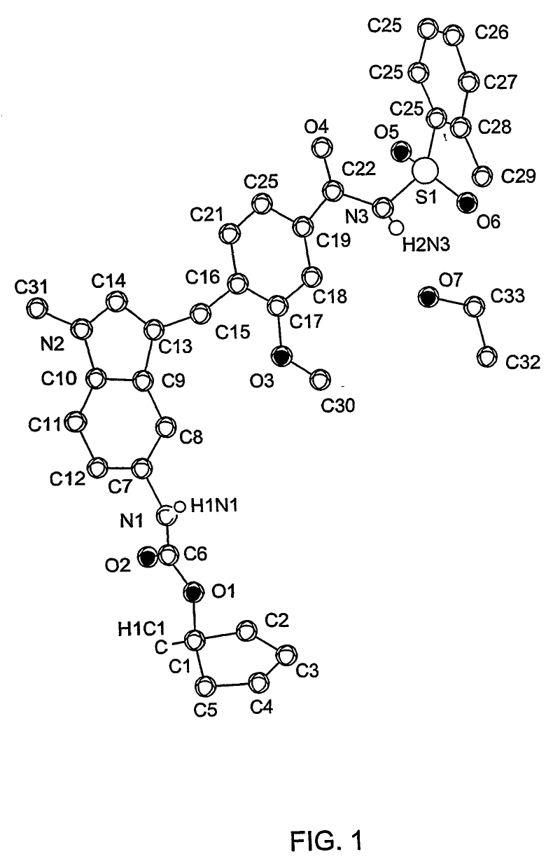

This invention also relates to a crystalline zafirlukast ethanolate having a single crystal x-ray crystallographic analysis, which yields atomic positions of all atoms relative to the origin of the unit cell as showed in Tables 2 through 6, and as represented in FIG. 1. Tables 2 through 6 list the parameters of atomic coordinates, and their isotropic thermal parameters, bond lengths, bond angles, anisotropic thermal parameters, and proton atom coordinates and their isotropic thermal parameters. The parameters presented in the tables are measured in units commonly used by those skilled in the art. These parameters define the absolute atomic arrangement in the crystal structure of zafirlukast ethanolate and this arrangement is depicted as the three dimensional structure in FIG. 1.

TABLE 2
Atomic coordinates (× 10) and equivalent isotropic
displacement parameters (Å² × 10³).
U(eq) is defined as one third of the trace of the
orthogonalized Uij tensor

| | x | y | z | U (eq) |
|---|---|---|---|---|
| S(1) | 3110 | 6437(2) | 3040 | 103(1) |
| O(1) | 5549(5) | −4791(7) | 3030(5) | 152(3) |
| O(2) | 6604(5) | −3637(7) | 2919(5) | 152(3) |
| O(3) | 3677(3) | 594(4) | 2035(4) | 91(2) |
| O(4) | 3468(3) | 6023(5) | 1631(3) | 104(2) |
| O(5) | 3829(4) | 7171(5) | 3098(4) | 135(3) |
| O(6) | 2944(4) | 6083(6) | 3728(3) | 132(2) |
| N(1) | 5320(7) | −3398(10) | 2140(9) | 138(5) |
| N(2) | 5409(6) | 250(8) | 98(5) | 102(2) |
| N(3) | 3222(5) | 5125(6) | 2629(5) | 84(2) |
| C(1) | 6144(9) | −5470(20) | 3693(9) | 186(8) |
| C(2) | 5997(9) | −4788(14) | 4313(13) | 182(6) |
| C(3) | 5380(11) | −5441(16) | 4552(7) | 193(5) |
| C(4) | 5228(11) | −6575(14) | 4126(8) | 191(6) |
| C(5) | 5810(13) | −6745(15) | 3759(14) | 226(9) |
| C(6) | 5913(10) | −3904(12) | 2729(7) | 125(4) |
| C(7) | 5397(6) | 2462(8) | 1604(6) | 95(3) |
| C(8) | 4737(5) | −1665(8) | 1345(5) | 92(2) |
| C(9) | 4766(5) | −789(8) | 803(5) | 83(2) |
| C(10) | 5512(7) | −719(9) | 619(6) | 94(3) |
| C(11) | 6168(5) | −1486(11) | 935(7) | 107(3) |
| C(12) | 6099(6) | −2396(9) | 1419(7) | 109(3) |
| C(13) | 4238(5) | 175(8) | 389(5) | 81(2) |
| C(14) | 4645(8) | 746(8) | −28(5) | 104(3) |
| C(15) | 3390(5) | 459(7) | 438(4) | 85(2) |
| C(16) | 3385(4) | 1636(7) | 852(5) | 69(2) |
| C(17) | 3535(4) | 1682(8) | 1654(5) | 75(2) |
| C(18) | 3500(5) | 2788(9) | 2028(4) | 79(2) |
| C(19) | 3405(4) | 3831(7) | 1605(5) | 69(2) |
| C(20) | 3311(5) | 3829(7) | 831(5) | 80(2) |
| C(21) | 3291(5) | 2707(9) | 472(4) | 89(2) |
| C(22) | 3385(5) | 5063(9) | 1920(6) | 82(2) |
| C(23) | 2237(5) | 7146(8) | 2369(5) | 88(2) |
| C(24) | 2374(7) | 8168(9) | 2015(6) | 117(3) |
| C(25) | 1696(9) | 8795(9) | 1501(7) | 135(4) |
| C(26) | 932(8) | 8332(12) | 1383(7) | 131(4) |
| C(27) | 808(7) | 7311(12) | 1733(8) | 119(3) |
| C(28) | 1463(7) | 6652(9) | 2247(6) | 105(3) |
| C(29) | 1256(6) | 5549(7) | 2630(5) | 110(3) |
| C(30) | 3846(6) | 605(7) | 2849(5) | 119(3) |
| C(31) | 6011(7) | 717(10) | 236(7) | 156(4) |
| O(7) | 7772(4) | −1868(5) | 3391(4) | 109(2) |
| C(32) | 7988(9) | −2477(13) | 4673(9) | 232(8) |
| C(33) | 7836(13) | −1461(14) | 4181(8) | 260(9) |

TABLE 3
Bond lengths (Å)

| | | | |
|---|---|---|---|
| S(1)—O(6) | 1.433(7) | C(15)—C(16) | 1.505(9) |
| S(1)—O(5) | 1.453(6) | C(15)—H(15A) | 0.9699 |
| S(1)—N(3) | 1.670(7) | C(15)—H(15B) | 0.9702 |
| S(1)—C(23) | 1.773(9) | C(16)—C(21) | 1.352(9) |
| O(1)—C(6) | 1.372(13) | | |
| O(1)—C(1) | 1.499(13) | C(16)—C(17) | 1.401(9) |
| O(2)—C(6) | 1.160(14) | C(17)—C(18) | 1.409(10) |
| O(3)—C(17) | 1.367(8) | C(18)—C(19) | 1.364(9) |
| O(3)—C(30) | 1.416(9) | C(18)—H(18) | 0.9300 |
| O(4)—C(22) | 1.213(8) | C(19)—C(20) | 1.367(9) |
| N(1)—C(6) | 1.334(15) | C(19)—C(22) | 1.480(11) |
| N(1)—C(7) | 1.460(14) | C(20)—C(21) | 1.396(10) |
| N(1)—H(1N1) | 0.58(6) | C(20)—H(20) | 0.9299 |
| N(2)—C(14) | 1.372(10) | C(21)—H(21) | 0.9301 |
| N(2)—C(10) | 1.402(10) | C(23)—C(24) | 1.359(11) |
| N(2)—C(31) | 1.460(10) | C(23)—C(28) | 1.388(11) |
| N(3)—C(22) | 1.417(10) | C(24)—C(25) | 1.412(13) |
| N(3)—H(2N3) | 0.78(5) | C(24)—H(24) | 0.9300 |
| C(1)—C(2) | 1.449(19) | C(25)—C(26) | 1.360(14) |
| C(1)—C(5) | 1.54(2) | C(25)—H(25) | 0.9300 |
| C(1)—H(5C1) | 1.10(8) | C(26)—C(27) | 1.346(13) |
| C(2)—C(3) | 1.464(18) | C(26)—H(26) | 0.9301 |
| C(2)—H(2A) | 0.9704 | C(27)—C(28) | 1.405(13) |
| C(2)—H(2B) | 0.9696 | C(27)—H(27) | 0.9298 |
| C(3)—C(4) | 1.449(16) | C(28)—C(29) | 1.505(11) |
| C(3)—H(3A) | 0.9698 | C(29)—H(29A) | 0.9600 |
| C(3)—H(3B) | 0.9698 | C(29)—H(29B) | 0.9600 |
| C(4)—C(5) | 1.39(2) | C(29)—H(29C) | 0.9600 |
| C(4)—H(4A) | 0.9696 | C(30)—H(30A) | 0.9600 |
| C(4)—H(4B) | 0.9703 | C(30)—H(30B) | 0.9600 |
| C(5)—H(5A) | 0.9696 | | |
| C(5)—H(5B) | 0.9704 | C(30)—H(30C) | 0.9600 |
| C(7)—C(12) | 1.360(12) | C(31)—H(31A) | 0.9600 |
| C(7)—C(8) | 1.389(10) | C(31)—H(31B) | 0.9600 |
| C(8)—C(9) | 1.395(10) | C(31)—H(31C) | 0.9600 |
| C(8)—H(8) | 0.9300 | O(7)—C(33) | 1.479(9) |
| C(9)—C(10) | 1.433(11) | O(7)—H(7O7) | 1.08(13) |
| C(9)—C(13) | 1.440(10) | C(32)—C(33) | 1.405(9) |
| C(10)—C(11) | 1.375(12) | C(32)—H(32A) | 0.9600 |
| C(11)—C(12) | 1.369(13) | C(32)—H(32B) | 0.9600 |
| C(12)—H(12) | 0.9299 | C(32)—H(32C) | 0.9600 |
| C(13)—C(14) | 1.350(11) | C(33)—H(33A) | 0.9704 |
| C(13)—C(15) | 1.525(10) | C(33)—H(33B) | 0.9694 |
| C(14)—H(14) | 0.9301 | C(11)—H(11) | 0.9300 |

TABLE 4
Bond Angles (°)

| | | | |
|---|---|---|---|
| O(6)—S(1)—O(5) | 120.2(4) | O(6)—S(1)—N(3) | 104.1(4) |
| O(5)—S(1)—N(3) | 107.0(4) | O(6)—S(1)—C(23) | 110.8(4) |
| O(5)—S(1)—C(23) | 108.0(4) | N(3)—S(1)—C(23) | 105.7(4) |
| C(6)—O(1)—C(1) | 113.5(11) | C(17)—O(3)—C(30) | 117.8(6) |
| C(6)—N(1)—C(7) | 128.3(11) | C(6)—N(1)—H(1N1) | 137(8) |
| C(7)—N(1)—H(1N1) | 93(8) | C(14)—N(2)—C(10) | 108.3(8) |
| C(14)—N(2)—C(31) | 124.3(10) | C(10)—N(2)—C(31) | 127.3(10) |
| C(22)—N(3)—S(1) | 122.7(7) | C(22)—N(3)—H(2N3) | 112(4) |
| S(1)—N(3)—H(2N3) | 118(4) | C(2)—C(1)—O(1) | 97.0(16) |
| C(2)—C(1)—C(5) | 104.2(13) | O(1)—C(1)—C(5) | 110.3(15) |
| C(2)—C(1)—H(5C1) | 121(4) | O(1)—C(1)—H(5C1) | 103(4) |
| C(5)—C(1)—H(5C1) | 119(4) | C(1)—C(2)—C(3) | 108.1(14) |
| C(1)—C(2)—H(2A) | 109.9 | C(3)—C(2)—H(2A) | 110.2 |
| C(1)—C(2)—H(2B) | 110.0 | C(3)—C(2)—H(2B) | 110.2 |
| H(2A)—C(2)—H(2B) | 108.4 | C(4)—C(3)—C(2) | 106.6(13) |
| C(4)—C(3)—H(3A) | 110.5 | C(2)—C(3)—H(3A) | 110.3 |
| C(4)—C(3)—H(3B) | 110.5 | C(2)—C(3)—H(3B) | 110.3 |
| H(3A)—C(3)—H(3B) | 108.7 | C(5)—C(4)—C(3) | 110.1(16) |
| C(5)—C(4)—H(4A) | 109.7 | C(3)—C(4)—H(4A) | 109.6 |
| C(5)—C(4)—H(4B) | 109.6 | C(3)—C(4)—H(4B) | 109.6 |
| H(4A)—C(4)—H(4B) | 108.2 | C(4)—C(5)—C(1) | 105.1(14) |
| C(4)—C(5)—H(5A) | 110.7 | C(1)—C(5)—H(5A) | 110.8 |
| C(4)—C(5)—H(5B) | 110.6 | C(1)—C(5)—H(5B) | 110.7 |
| H(5A)—C(5)—H(5B) | 108.8 | O(2)—C(6)—N(1) | 124.6(14) |
| O(2)—C(6)—O(1) | 128.4(12) | N(1)—C(6)—O(1) | 107.0(13) |
| C(12)—C(7)—C(8) | 125.1(9) | C(12)—C(7)—N(1) | 119.5(10) |
| C(8)—C(7)—N(1) | 115.4(10) | C(7)—C(8)—C(9) | 117.4(8) |
| C(7)—C(8)—H(8) | 121.3 | C(9)—C(8)—H(8) | 121.3 |
| C(8)—C(9)—C(10) | 116.8(8) | C(8)—C(9)—C(13) | 135.9(9) |
| C(10)—C(9)—C(13) | 107.2(9) | C(11)—C(10)—N(2) | 130.8(10) |
| C(11 )—C(10)—C(9) | 122.9(9) | N(2)—C(10)—C(9) | 106.3(9) |
| C(12)—C(11)—C(10) | 119.1(9) | C(12)—C(11)—H(11) | 120.4 |
| C(10)—C(11)—H(11) | 120.5 | C(7)—C(12)—C(11) | 118.4(9) |
| C(7)—C(12)—H(12) | 120.8 | C(11)—C(12)—H(12) | 120.8 |
| C(14)—C(13)—C(9) | 106.6(8) | C(14)—C(13)—C(15) | 128.6(9) |
| C(9)—C(13)—C(15) | 124.8(8) | C(13)—C(14)—N(2) | 111.6(8) |
| C(13)—C(14)—H(14) | 124.2 | N(2)—C(14)—H(14) | 124.2 |
| C(16)—C(15)—C(13) | 111.8(6) | C(16)—C(15)—H(15A | 109.3 |
| C(13)—C(15)— | 109.3 | C(16)—C(15)—H(15B) | 109.2 |
| H(15A) | | | |
| C(13)—C(15)— | 109.2 | H(15A)—C(15)— | 107.9 |
| H(15B) | | H(15B) | |
| C(21)—C(16)—C(17) | 117.0(8) | C(21)—C(16)—C(15) | 121.1(8) |
| C(17)—C(16)—C(15) | 121.8(8) | O(3)—C(17)—C(16) | 116.1(8) |
| O(3)—C(17)—C(18) | 122.9(8) | C(16)—C(17)—C(18) | 121.0(8) |
| C(19)—C(18)—C(17) | 118.2(7) | C(19)—C(18)—H(18) | 120.9 |
| C(17)—C(18)—H(18) | 120.9 | C(18)—C(19)—C(20) | 122.3(7) |
| C(18)—C(19)—C(22) | 124.7(8) | C(20)—C(19)—C(22) | 113.1(8) |
| C(19)—C(20)—C(21) | 117.6(7) | C(19)—C(20)—H(20) | 121.2 |
| C(21)—C(20)—H(20) | 121.2 | C(16)—C(21)—C(20) | 123.6(7) |
| C(16)—C(21)—H(21) | 118.2 | C(20)—C(21)—H(21) | 118.2 |
| O(4)—C(22)—N(3) | 116.3(8) | O(4)—C(22)—C(19) | 127.7(9) |
| N(3)—C(22)—C(19) | 115.9(8) | C(24)—C(23)—C(28) | 124.0(9) |
| C(24)—C(23)—S(1) | 116.9(8) | C(28)—C(23)—S(1) | 119.0(8) |
| C(23)—C(24)—C(25) | 119.1(10) | C(23)—C(24)—H(24) | 120.4 |
| C(25)—C(24)—H(24) | 120.5 | C(26)—C(25)—C(24) | 117.6(10) |
| C(26)—C(25)—H(25) | 121.2 | C(24)—C(25)—H(25) | 121.2 |
| C(27)—C(26)—C(25) | 122.4(10) | C(27)—C(26)—H(26) | 118.8 |
| C(25)—C(26)—H(26) | 118.8 | C(26)—C(27)—C(28) | 122.1(10) |
| C(26)—C(27)—H(27) | 118.9 | C(28)—C(27)—H(27) | 118.9 |
| C(23)—C(28)—C(27) | 114.7(9) | C(23)—C(28)—C(29) | 127.3(10) |
| C(27)—C(28)—C(29) | 117.9(10) | C(28)—C(29)—H(29A) | 109.5 |
| C(28)—C(29)— | 109.5 | H(29A)—C(29)— | 109.5 |
| H(29B) | | H(29B) | |
| C(28)—C(29)— | 109.5 | H(29A)—C(29)— | 109.5 |
| H(29C) | | H(29C) | |
| H(29B)—C(29)— | 109.5 | O(3)—C(30)—H(30A) | 109.5 |
| H(29C) | | | |
| O(3)—C(30)—H(30B) | 109.5 | H(30A)—C(30)— | 109.5 |
| | | H(30B) | |
| O(3)—C(30)—H(30C) | 109.5 | H(30A)—C(30)— | 109.5 |
| | | H(30C) | |
| H(30B)—C(30)— | 109.5 | N(2)—C(31)—H(31A) | 109.5 |
| H(30C) | | | |
| N(2)—C(31)—H(31B) | 109.5 | H(31A)—C(31)— | 109.5 |
| | | H(31B) | |
| N(2)—C(31)—H(31C) | 109.5 | H(31A)—C(31)— | 109.5 |
| | | H(31C) | |
| H(31B)—C(31)— | 109.5 | C(33)—O(7)—H(7O7) | 118(6) |
| H(31C) | | | |
| C(33)—C(32)— | 109.4 | C(33)—C(32)—H(32B) | 109.5 |
| H(32A) | | | |
| H(32A)—C(32)— | 109.5 | C(33)—C(32)—H(32C) | 109.4 |
| H(32B) | | | |
| H(32A)—C(32)— | 109.5 | H(32B)—C(32)— | 109.5 |
| H(32C) | | H(32C) | |
| C(32)—C(33)—O(7) | 108.6(12) | C(32)—C(33)—H(33A) | 109.9 |
| O(7)—C(33)—H(33A) | 109.9 | C(32)—C(33)—H(33B) | 110.0 |
| O(7)—C(33)—H(33B) | 110.0 | H(33A)—C(33)— | 108.4 |
| | | H(33B) | |

TABLE 5
Anisotropic displacement parameters (Å² × 10³)

| | U₁₁ | U₂₂ | U₃₃ | U₂₃ | U₁₃ | U₁₂ |
|---|---|---|---|---|---|---|
| S(1) | 104(2) | 89(2) | 104(2) | −22(2) | 21(2) | −9(2) |
| O(1) | 105(5) | 154(6) | 174(7) | 49(5) | 15(5) | −18(5) |
| O(2) | 77(4) | 159(6) | 186(7) | 7(5) | 0(5) | −30(5) |
| O(3) | 121(4) | 56(3) | 94(4) | 10(3) | 35(3) | −4(3) |
| O(4) | 107(4) | 81(4) | 134(5) | 25(4) | 55(4) | −7(3) |
| O(5) | 92(5) | 81(4) | 197(7) | −25(4) | 1(4) | −32(3) |
| O(6) | 181(7) | 117(5) | 98(4) | −24(4) | 47(4) | −10(4) |
| N(1) | 76(8) | 114(8) | 215(13) | 24(8) | 37(9) | −19(7) |
| N(2) | 104(6) | 109(6) | 110(6) | −33(5) | 59(5) | −33(5) |
| N(3) | 90(5) | 54(5) | 113(6) | −11(4) | 40(4) | −22(4) |
| C(1) | 106(11) | 340(30) | 112(11) | 93(14) | 36(9) | 56(14) |
| C(2) | 132(12) | 187(14) | 214(17) | 41(13) | 41(12) | −28(9) |
| C(3) | 250(17) | 198(14) | 144(11) | −18(11) | 84(11) | −27(13) |
| C(4) | 267(19) | 146(12) | 127(9) | −34(9) | 20(11) | −62(12) |
| C(5) | 320(20) | 121(14) | 300(20) | 4(12) | 190(19) | 54(13) |
| C(6) | 101(10) | 135(11) | 130(10) | 7(8) | 26(8) | 11(9) |
| C(7) | 71(7) | 87(7) | 123(7) | 11(6) | 28(5) | −11(5) |
| C(8) | 86(6) | 88(6) | 101(6) | 2(5) | 31(5) | −8(5) |
| C(9) | 80(6) | 93(6) | 88(6) | −16(5) | 41(5) | −6(5) |
| C(10) | 106(9) | 94(7) | 99(7) | −4(6) | 56(6) | −18(6) |
| C(11) | 64(6) | 110(8) | 148(8) | −48(7) | 38(6) | −22(6) |
| C(12) | 63(6) | 85(7) | 181(9) | −4(6) | 46(6) | −12(5) |
| C(13) | 77(6) | 77(6) | 100(6) | −12(5) | 42(5) | −4(5) |
| C(14) | 113(8) | 92(6) | 114(8) | 1(5) | 45(6) | −9(6) |
| C(15) | 78(6) | 85(6) | 85(6) | −26(4) | 17(4) | −2(4) |
| C(16) | 76(5) | 64(5) | 66(6) | 6(5) | 20(4) | 4(4) |
| C(17) | 69(5) | 75(6) | 87(6) | 6(6) | 33(4) | 4(4) |
| C(18) | 79(6) | 92(7) | 68(5) | −2(5) | 29(4) | −19.5 |
| C(19) | 65(5) | 57(6) | 93(6) | 12(5) | 37(4) | 3(4) |
| C(20) | 89(6) | 84(6) | 74(6) | 23(5) | 35(4) | 3(4) |
| C(21) | 100(6) | 103(7) | 69(5) | 0(6) | 33(4) | 5(5) |
| C(22) | 79(6) | 80(7) | 98(7) | −2(5) | 45(5) | −4(5) |
| C(23) | 82(7) | 75(6) | 101(6) | −24(5) | 22(5) | −6(5) |
| C(24) | 152(10) | 71(7) | 133(8) | −5(6) | 53(8) | −10(6) |
| C(25) | 135(10) | 116(9) | 141(9) | 30(7) | 30(7) | −7(8) |
| C(26) | 94(8) | 121(9) | 147(9) | 15(8) | 0(7) | 7(7) |
| C(27) | 96(8) | 114(8) | 160(10) | −22(7) | 59(8) | −6(7) |
| C(28) | 87(7) | 110(8) | 117(7) | −24(6) | 34(6) | −21(7) |
| C(29) | 121(7) | 87(7) | 126(8) | −31(6) | 46(6) | −34(5) |
| C(30) | 175(9) | 97(7) | 73(6) | 15(5) | 25(6) | −15(6) |
| C(31) | 149(9) | 165(10) | 206(11) | −3(8) | 129(8) | −34(8) |
| O(7) | 136(5) | 109(5) | 91(4) | −12(3) | 49(3) | −27(4) |
| C(32) | 236(16) | 165(11) | 206(14) | 45(11) | −42(12) | −40(11) |
| C(33) | 400(30) | 230(20) | 181(15) | 15(14) | 146(17) | 22(19) |

TABLE 6
Hydrogen coordinates (× 10⁴) and isotropic
displacement parameters (Å² × 10³)

| | x | y | z | U(eq) |
|---|---|---|---|---|
| H(2A) | 5798 | −3982 | 4133 | 218 |
| H(2B) | 6504 | −4709 | 4749 | 218 |
| H(3A) | 5585 | −5593 | 5107 | 232 |
| H(3B) | 4877 | −4971 | 4429 | 232 |
| H(4A) | 5255 | −7241 | 4481 | 230 |
| H(4B) | 4680 | −6563 | 3742 | 230 |
| H(5A) | 5559 | −7101 | 3249 | 271 |
| H(5B) | 6250 | −7273 | 4065 | 271 |
| H(8) | 4293 | −1713 | 1526 | 110 |
| H(11) | 6652 | −1388 | 821 | 128 |
| H(12) | 6521 | −2955 | 1616 | 130 |
| H(14) | 4433 | 1395 | −359 | 125 |
| H(15A) | 2992 | 502 | −83 | 102 |
| H(15B) | 3224 | −192 | 711 | 102 |
| H(18) | 3541 | 2807 | 2549 | 94 |
| H(20) | 3262 | 4549 | 555 | 96 |
| H(21) | 3208 | 2696 | −58 | 107 |
| H(24) | 2908 | 8451 | 2111 | 141 |
| H(25) | 1769 | 9499 | 1251 | 162 |
| H(26) | 477 | 8736 | 1048 | 157 |
| H(27) | 273 | 7033 | 1631 | 143 |
| H(29A) | 686 | 5347 | 2384 | 165 |
| H(29B) | 1359 | 5713 | 3170 | 165 |
| H(29C) | 1592 | 4881 | 2580 | 165 |
| H(30A) | 4046 | −177 | 3059 | 178 |
| H(30B) | 3350 | 789 | 2957 | 178 |
| H(30C) | 4255 | 1209 | 3082 | 178 |
| H(31A) | 6382 | 80 | −258 | 234 |
| H(31B) | 6317 | 1366 | 82 | 234 |
| H(31C) | 5730 | 1014 | −750 | 234 |
| H(32A) | 8209 | −2216 | 5204 | 347 |
| H(32B) | 8377 | −3000 | 4557 | 347 |
| H(32C) | 7483 | −2908 | 4595 | 347 |
| H(33A) | 8281 | −881 | 4371 | 312 |
| H(33B) | 7327 | −1072 | 4170 | 312 |
| H(1N1) | 4990(40) | −3510(60) | 1960(40) | 30(20) |
| H(2N3) | 2980(30) | 4560(50) | 2690(30) | 37(19) |
| H(5C1) | 6730(50) | −5390(60) | 3580(40) | 100(30) |
| H(7O7) | 7300(80) | −2510(110) | 3110(60) | 190(50) |

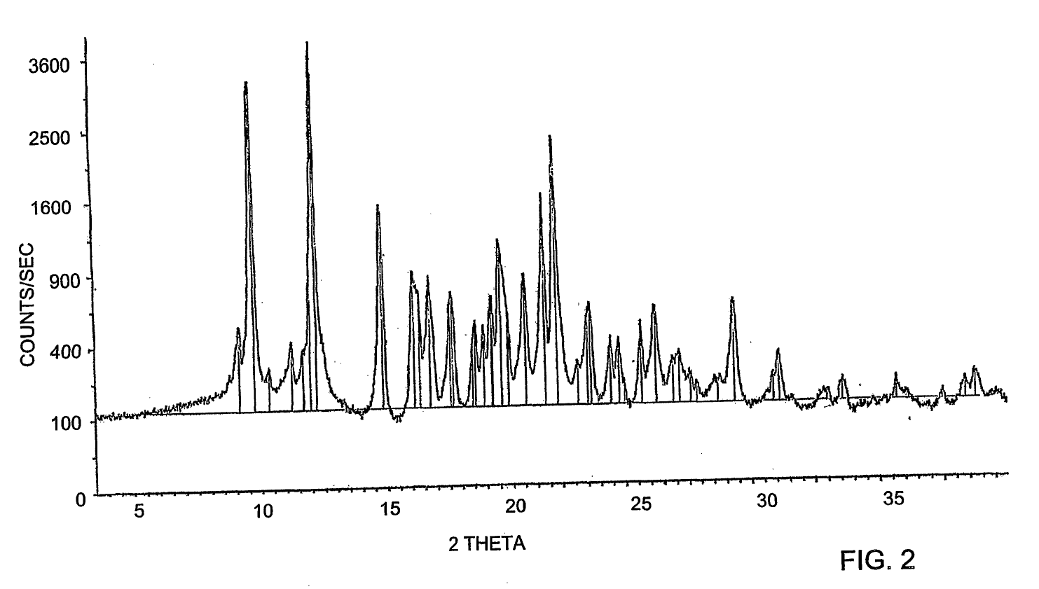

Zafirlukast ethanolate also give distinctive x-ray powder diffraction pattern, as depicted in FIG. 2. The pattern has characteristic peaks expressed in degrees 2θ at approximately 10.3, 12.7, 15.4, 18.3, 20.1, 21.9 and 22.4.

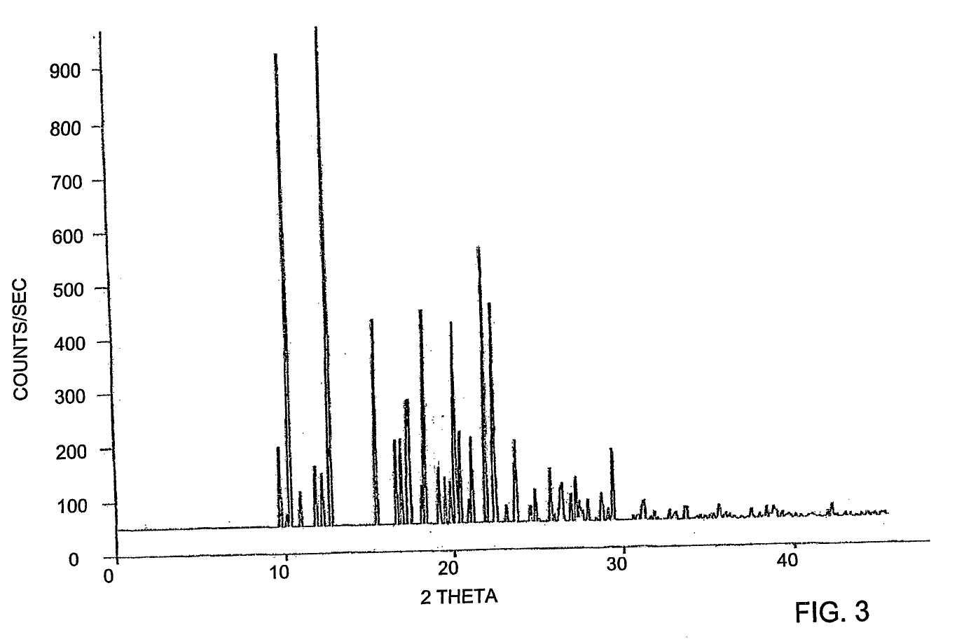

The results of a single crystal x-ray analysis are limited to, as the name implies, to one crystal placed in the x-ray beam. Crystallographic data on a large group of crystals provides powder x-ray diffraction. If the powder consists of a pure crystalline compound, a simple powder diagram is obtained. To compare the results of a single crystal analysis and a powder x-ray analysis, a simple calculation can be done converting the single crystal analysis and powder x-ray diagram. This conversion is possible because the single crystal experiment routinely determines the unit cell dimensions, space group, and atomic positions. These parameters provide a basis to calculate a perfect powder pattern. Comparing this calculated powder pattern and the powder pattern experimentally obtained from a large collection of crystals will confirm if the results of the two techniques are the same. This has been done for zafirlukast ethanolate and the results are graphically displayed in FIGS. 2 and 3 and in Table 7.

TABLE 7
Calculated from single crystal x-ray analysis versus experimental x-ray
powder diffraction pattern (λ = 1.5418 Å radiation)
wherein d represents the interplanar spacing and I/I₁ represents
the relative intensity:

| 2θ(°) | d(Å) | I/I₁ | h | k | l |
|---|---|---|---|---|---|
| 9.69 | 9.13 | .16 | 1 | 1 | 0 |
| 10.28 | 8.61 | .92 | 0 | 0 | 2 |
| 10.90 | 8.12 | .07 | 2 | 0 | 0 |
| 11.80 | 7.50 | .12 | 1 | 1 | 1 |
| 12.24 | 7.22 | .10 | 2 | 0 | −2 |
| 12.75 | 6.94 | 1.00 | 1 | 1 | −2 |
| 15.42 | 5.75 | .40 | 1 | 1 | 2 |
| 16.64 | 5.33 | .17 | 1 | 1 | −3 |
| 16.88 | 5.25 | .17 | 0 | 2 | 1 |
| 17.34 | 5.12 | .24 | 2 | 0 | 2 |
| 17.42 | 5.09 | .24 | 3 | 1 | −1 |
| 18.08 | 4.91 | .08 | 3 | 1 | −2 |
| 18.26 | 4.86 | .42 | 3 | 1 | 0 |
| 19.11 | 4.64 | .11 | 0 | 2 | 2 |
| 19.46 | 4.56 | .09 | 2 | 2 | 0 |
| 19.76 | 4.49 | .08 | 1 | 1 | 3 |
| 20.11 | 4.42 | .39 | 3 | 1 | −3 |
| 20.25 | 4.39 | .13 | 2 | 2 | −2 |
| 20.43 | 4.35 | .18 | 3 | 1 | 1 |
| 21.06 | 4.22 | .09 | 2 | 2 | 1 |
| 21.11 | 4.21 | .08 | 1 | 1 | −4 |
| 21.90 | 4.06 | .54 | 4 | 0 | 0 |
| 22.36 | 3.98 | .43 | 0 | 2 | 3 |
| 22.51 | 3.95 | .09 | 2 | 2 | −3 |
| 23.57 | 3.77 | .07 | 3 | 1 | 2 |
| 23.72 | 3.75 | .16 | 2 | 2 | 2 |
| 24.82 | 3.59 | .06 | 1 | 3 | 0 |
| 25.66 | 3.47 | .10 | 2 | 2 | 4 |
| 26.26 | 3.39 | .07 | 0 | 2 | 4 |
| 26.35 | 3.38 | .06 | 4 | 2 | −1 |
| 26.89 | 3.32 | .05 | 3 | 1 | −5 |
| 27.24 | 3.27 | .05 | 4 | 0 | 2 |
| 28.66 | 3.11 | .06 | 5 | 1 | 0 |
| 29.39 | 3.04 | .06 | 1 | 1 | 5 |
| 29.43 | 3.035 | .06 | 2 | 2 | −5 |

FIG. 2 shows an experimentally derived powder x-ray diffraction pattern of zafirlukast ethanolate and FIG. 3 corresponds to the x-ray diffraction derived from the single crystal x-ray data. The peak overlap indicates that the two techniques yield the same results. The primary powder x-ray diffraction peaks provide an unambiguous description of the crystalline state of zafirlukast ethanolate.

A pure crystalline organic compound has, in general, a definite melting point range. The melting point is defined as the point at which the sample is entirely in the liquid phase. The crystalline zafirlukast ethanolate has characteristic melting point range determined by the capillary method from 132 to 133° C. (dec.).

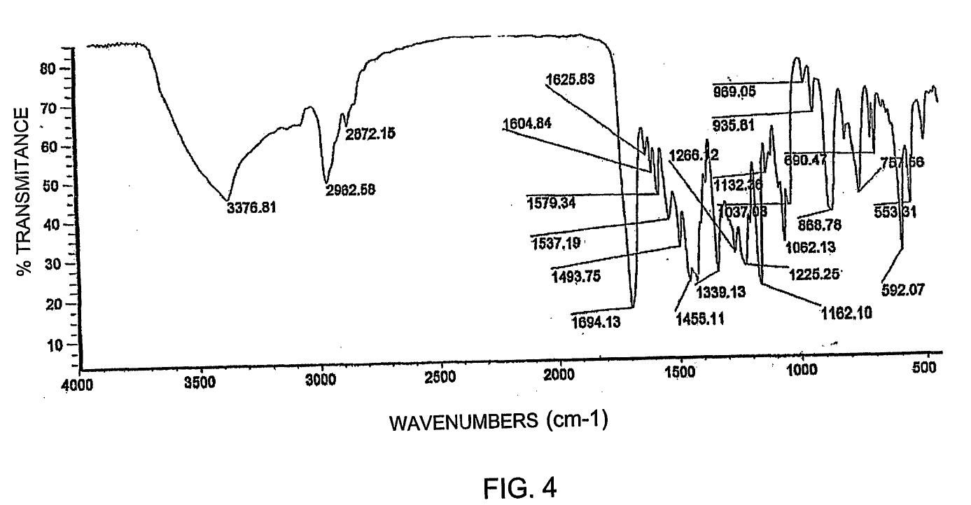

The crystalline zafirlukast ethanolate was further characterized by an infrared absorption spectrum in potassium bromide pattern substantially as depicted in FIG. 4.

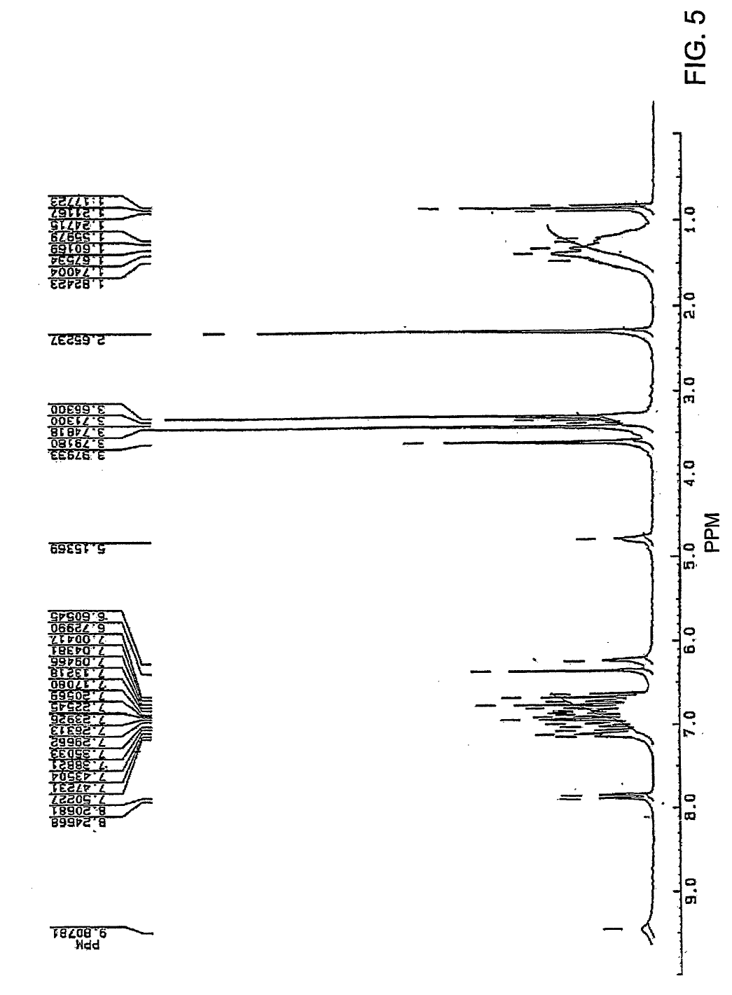

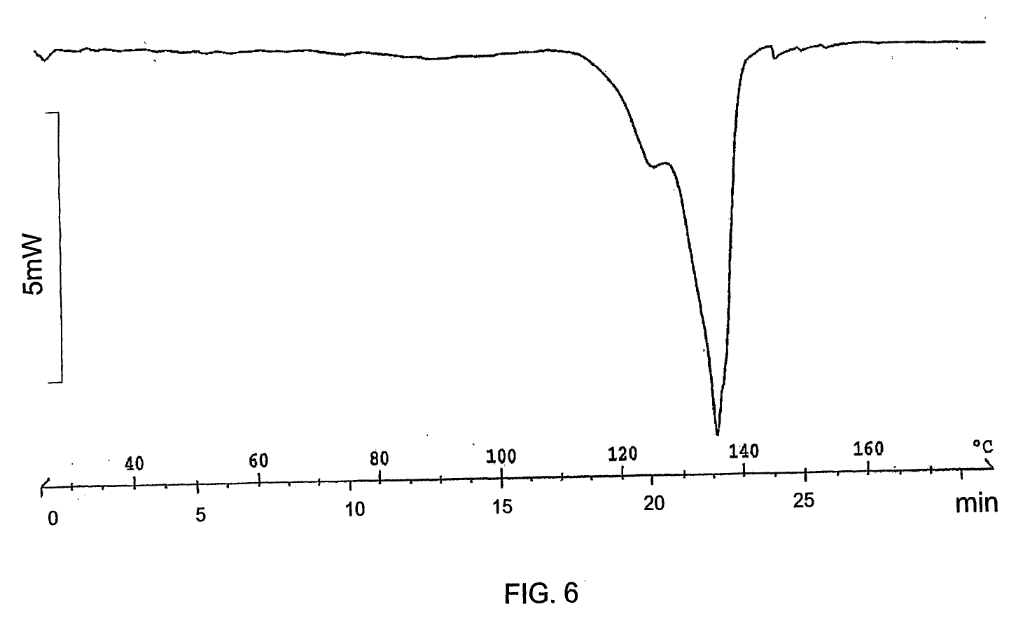

Zafirlukast ethanolate was further characterized by a ¹H nuclear magnetic resonance spectrum in chloroform-d having characteristic absorption bands at approximately δ (ppm) 1.2 (triplet, J 7 Hz) and 3.7 (quartet, J 7 Hz) and a ¹³C nuclear magnetic resonance spectrum in chloroform-d having characteristic absorption bands at approximately δ (ppm) 17 and 57. The ¹H NMR spectrum of zafirlukast ethanolate is showed in FIG. 5 and the DSC thermogram is showed in FIG. 6.

According to another aspect of the invention, there are disclosed methods for the preparation of zafirlukast ethanolate. According to one method, zafirlukast ethanolate is prepared by:

a) dissolving zafirlukast in solvent A;

b) adding absolute or aqueous ethanol to the zafirlukast solution obtained in step a);

c) optionally, removing partially or completely solvent A and excess of ethanol so that crystallization of the zafirlukast ethanolate begins and a suspension or slurry is formed; and

d) isolating the crystals of zafirlukast ethanolate.

More specifically, zafirlukast ethanolate is prepared by dissolving zafirlukast in an organic solvent having similar or higher solubility of zafirlukast, as compared to absolute ethanol. This solvent is termed hereinafter “solvent A”. Absolute or aqueous ethanol is then added to the zafirlukast solution. Subsequently, solvent A and excess of ethanol are removed partially or completely from the mixture at a temperature below the melting point of zafirlukast ethanolate so that crystallization of zafirlukast ethanolate begins and a suspension or slurry is formed. The crystals of zafirlukast ethanolate are then isolated from the suspension or slurry. Alternatively, the ethanolate may be prepared by rapidly evaporating, preferably by spray drying, of a solution of zafirlukast in a mixture of solvent A and absolute or aqueous ethanol at a temperature below the melting point of zafirlukast ethanolate. The method may further comprise the step of maintaining the suspension or slurry at a temperature from about −5 to about 80° C. for about 1 to about 18 hours. Preferably, the suspension is cooled to below 20° C. prior to isolating the crystals of zafirlukast ethanolate. Preferably, the method further comprises the step of drying the crystals of zafirlukast ethanolate at a temperature below the melting point of the zafirlukast ethanolate.

Solvents which function as solvent A are for example chloroorganic solvents, ketones or nitriles. Preferably, solvent A is dichloromethane, chloroform, acetone, methyl ethyl ketone (MEK), methyl isopropyl ketone, acetonitrile or mixture thereof.

According to another method, zafirlukast ethanolate may be prepared using the following steps:

a) mixing of zafirlukast with absolute or aqueous ethanol;

b) b) stiring the obtained mixture for a time sufficient to induce the transformation of zafirlukast to zafirlukast ethanolate; and

c) c) isolating the crystals of zafirlukast ethanolate.

The starting material zafirlukast used in this method consisted of Forms A, B or X prepared according to U.S. Pat. No. 5,993,859 or zafirlukast prepared according to J. Med. Chem., v. 33, 1781 (1990). The new methods of the invention may also be useful for purification of zafirlukast from related impurities.

Preferably, the methods further comprise the step of drying the crystals of zafirlukast ethanolate. Effective drying methods include vacuum oven drying, air oven or simple vacuum desiccator drying. The ethanolate is stable at ambient temperature and will tolerate vacuum drying. When vacuum oven drying is used, one must exercise caution to avoid breaking the ethanolate. Preferred drying conditions for the ethanol solvate are about 30 to about 70° C. vacuum oven drying and are about 30 to about 105° C. at air oven drying. For example, drying of zafirlukast ethanolate with mp 130 - 132° C. at 105±1° C. for 4 hours in an air oven resulted in less then 1% weight loss without substantial change of physical properties (including mp, ¹H NMR and infra-red spectra) of the solvate.

The ethanolate of this invention is particularly useful because it is stable under conditions of high relative humidity and elevated temperatures.

In accordance with the present invention, the new ethanolate of zafirlukast may be prepared as pharmaceutical compositions that are particularly useful for the treatment of disease in which leukotrienes are implicated, for example for the treatment of asthma. Such compositions comprise an effective amount of zafirlukast ethanolate together with pharmaceutically acceptable carriers and/or excipients.

It has been found that compositions according to the invention have acceptable physical stability, can be prepared reproducibly and have high bioavailability.

The composition according to the invention may be in any conventional form suitable for oral administration, for example in the form of a tablet, capsule, beadlet or powder. Preferably it is in the form of a tablet.

In the composition according to the invention, the active ingredient is conveniently present in an amount of from 1 to 90% by weight, based upon the total weight of the composition, for example from 10 to 50% by weight. However, the composition will more usually further comprise at least one pharmaceutically acceptable carrier.

Examples of suitable pharmaceutically acceptable carriers include, for example, sugar derivatives such as mannitol, lactose, sorbitol, glucose, sucrose, dextrose, fructose and xylitol, and cellulose derivatives such as microcrystalline cellulose, powdered cellulose and hydroxypropylmethylcellulose. Preferably the composition comprises a sugar derivative, especially lactose, and a cellulose derivative, especially microcrystalline cellulose. The amount of sugar derivative present may, for example be in the range of from 10 to 30% by weight based upon the total weight of the composition. The amount of cellulose derivative present may, for example, be in the range of from 25 to 70% by weight, based upon the total weight of the composition.

The composition may further comprise one or more processing adjuvants such as disintegrants, for example croscarmellose sodium, sodium starch glycolate and starch, and lubricants, for example magnesium stearate, stearic acid, talc and powdered vegetable stearine. The amount of disintegrant present may, for example, be in the range of from 1 to 10% by weight based upon the total weight of the composition. The amount of lubricant present may, for example, be in the range of from 0.25 to 2% by weight, based upon the total weight of the composition. The composition may further comprise polyvinylpyrrolidone in an amount of from 1 to 20% by weight based on the total weight of the composition, preferably from 2 to 6% by weight.

The composition may be prepared by mixing the ingredients according to a conventional method, for example by one of the granulation processes known in the art.

A possible process for preparing a pharmaceutical composition of zafirlukast ethanolate may comprise mixing a solution of zafirlukast in acetone or methyl ethyl ketone and absolute or aqueous ethanol with any other ingredients of the composition and drying the resultant mixture.

When the composition is in the form of a tablet, the weight of the tablet may conveniently be in the range of from 25 to 500 mg, such as from 50 to 250 mg, for example from 100 to 200 mg. The tablet may be uncoated or coated. The coating may be a conventional coating and may be applied by a conventional method.

The present invention also provides a method for the treatment of a disease state in which leukotrienes are implicated, such method comprising administering to a subject in need of such treatment, a therapeutically effective amount of zafirlukast ethanolate or a pharmaceutical composition thereof.

The dose of zafirlukast ethanolate to be administered to a patient in a composition according to the invention will depend upon the severity of the condition to be treated, the age and the weight of the patient. In general, the ethanolate will be administered at a dose in the range of from 0.1 to 10 mg/kg, for example from 0.2 to 5 mg/kg.

This invention will be better understood from the Examples that follow. However, the examples illustrate, but do not limit, the invention. Those skilled in the art will readily appreciate that the specific methods and results discussed are merely illustrative of the invention as described and claimed in the claims that follow thereafter.

### EXAMPLES

Experimental Details:

Hygroscopicities of zafirlukast ethanolate and zafirlukast Form A were compared by maintaining samples in controlled humidity chambers at 25° C. under 60% relative humidity and at 40° C. under 75% relative humidity for a period of two weeks, followed by Karl Fisher analysis of water content.

The ethanol content was determined by gas chromatography on a Hewlett-Packard 5890 gas chromatograph.

HPLC was carried out on a Merck-Hitachi Lachrom chromatographic system with UV detector.

Single crystal x-ray crystallographic analysis was performed on a Phillips PW 11000 diffractometer, (ω/2θ mode, graphite monochromator, MoK_{□} radiation.

Powder x-ray diffraction patterns were obtained by methods known in the art using a Philips analytical x-ray powder diffractometer for wide-angle x-ray diffraction (CuK_{α} radiation of γ=1.5418 Å, monochromator before detector, Pw3020 goniometer system). The Bragg-Brentano scheme was used for beam focusing.

¹H and ¹³C NMR spectra were recorded on a Bruker AM-200 (200 MHz) and Bruker AM-400 (400 MHz) instruments using CDCl₃ as a solvent.

Melting points were determined in open capillary tubes with Electrothermal IA 9300 Digital capillary melting point apparatus and are uncorrected. The melting points of zafirlukast ethanolate generally depend upon their level of purity. Typically, zafirlukast ethanolate has been found to have a melting point between 120 and 140° C., for example from 125 to 135° C.

Infrared spectra were recorded on a Nicolet Impact 410 FT-IR spectrophotometer using a 0.5% dispersion of sample material in a potassium bromide disk over the wave number range 4000 to 400 cm⁻¹.

DSC graphs were recorded on a Mettler DSC 30 Differential Scanning Calorimeter.

Thermo gravimetric analysis (TGA). Measurements were performed using TGA 2050 Thermo gravimetric Analyzer (TA Instruments Inc.).

### Example 1

Preparation of Crystalline Zafirlukast Ethanolate

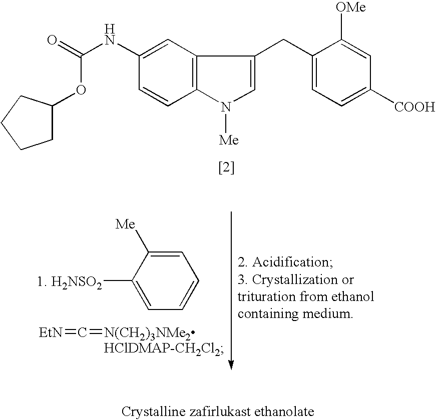

A mixture of 4-[[5-[[(cyclopentyloxy)carbonyl]amino]-1-methyl-1H-indol-3-yl]methyl]-3-methoxybenzoic acid [2] (700.0 g, 1.66 mol), o-toluenesulfonamide (297.9 g, 1.74 mol), 4-(dimethylamino)pyridine (DMAP) (212.5 g, 1.74 mol), 1-[3-(dimethylamino)propyl]-3-ethylcarbodiimide hydrochloride (333.5 g, 1.74 mol) and dichloromethane (3.5 L) was stirred for 24 hours at RT under inert atmosphere. 5% Hydrochloric acid (2 L) was added dropwise to the stirred mixture at 5-10° C. The obtained mixture was stirred for 20 min. The organic layer was separated, washed with water (1 L), quickly dried over anhydrous sodium sulfate (100 g) and passed through a short Silica gel (300 g) column, washing with dichloromethane (500 mL). The filtrate was concentrated under reduced pressure to the volume of 2 L, heated to reflux and absolute ethanol (5 L) was added to the solution. Dichloromethane was distilled off from the stirred mixture through a 10 cm Vigreux distilling column equipped with a distillation head until the temperature of vapor in the head of the column reached 78° C. The resulting mixture was kept overnight at room temperature. The precipitated crystals were filtered off, washed on filter with ice cold absolute ethanol (2×500 mL), dried under reduced pressure at 60° C. (water bath) and dissolved in hot dichloromethane (3.5 L). The solution was concentrated under reduced pressure to the volume of 2 L, heated to reflux and absolute ethanol (5 L) was added to the solution. Dichloromethane was distilled off from the stirred mixture through a 10 cm Vigreux distilling column equipped with a distillation head until the temperature of vapor in the head of the column was reached 78° C. The resulting mixture was kept overnight at the room temperature. The precipitated crystals were filtered off, washed on filter with ice cold absolute ethanol (2×500 mL) and dried under reduced pressure at 60° C. (water bath) to give 835.2 g (80.4% yield) of crystalline zafirlukast ethanolate as white powder with mp 132-133° C. (dec.) and 99.8 % purity by HPLC. Anal. (C₃₃H₃₉N₃O₇S) C, H, N.

### Example 2

Preparation of Crystalline Zafirlukast Ethanolate from Zafirlukast Forms A, B and X, Prepared According to U.S. Pat. No. 5,993,859 and Zafirlukast, Prepared According to J. Med. Chem., v. 33, 1781(1990).

5.0 g of zafirlukast Forms A, B or X, prepared according to U.S. Pat. No. 5,993,859 or zafirlukast, prepared according to J. Med. Chem., v. 33, 1781 (1990) were dissolved in hot acetone (50 mL). The solution was concentrated under reduced pressure to the volume of about 20 mL, heated to reflux and absolute ethanol (100 mL) was added to the solution. Acetone was distilled off from the stirred mixture through a 10 cm Vigreux column equipped with a distillation head until the temperature of vapor in the head of the column reached 78° C. The resulting mixture was kept overnight at the room temperature. The precipitated crystals were filtered off, washed on filter with ice cold absolute ethanol (2×5 mL) and dried under reduced pressure at 60° C. (water bath) to give crystalline zafirlukast ethanolate with mp 130-132° C. Drying of the crystalline zafirlukast ethanolate for 4 hours in an at air oven at 105±1° C. led to the loss of 0.86% weight without substantial change of physical properties.

### Example 3

Preparation of Crystalline Zafirlukast Ethanolate from Zafirlukast, Prepared According to J. Med. Chem., v. 33, 1781 (1990), in Absolute Ethanol.

A mixture of zafirlukast (5.0 g), prepared according to J. Med. Chem., v. 33, 1781 (1990), and absolute ethanol (50 mL) was stirred for 2 hours under reflux conditions, kept overnight at room temperature and for 2 hours at 0-5° C. The precipitated crystals were filtered off, washed on filter with ice cold absolute Ethanol (2×5 mL) and dried under reduced pressure to give zafirlukast ethanolate with mp 130-132° C. ¹H NMR (CDCl₃, δ, ppm): 1.22 (t, J 7.05 Hz, 3H), 1.45-1.87 (m, 8H), 2.66 (s, 3H), 3.67 (s, 3H), 3.73 (q, J 7.05 Hz, 4H), 3.79 (s, 3H), 3.98 (s, 2H), 5.08-5.23 (m, 1H), 6.58 (s, 1H), 6.73 (s, 1H), 7.01-7.51 (m, 9H), 8.23 (d, J 7.52 Hz, 1H), 9.67 (s, 1H).

### Example 4

Preparation of Crystalline Zafirlukast Ethanolate by Re-Slurry of Zafirlukast, Prepared According to J. Med. Chem., v. 33, 1781 (1990), in 95 % Ethanol.

A mixture of zafirlukast (5.0 g), prepared according to J. Med. Chem. 33:1781 (1990), and 95 % ethanol (50 mL) was stirred for 2 hours under reflux conditions, kept overnight at room temperature and for 2 hours at 0-5° C. The precipitated crystals were filtered off, washed on filter with ice-cold absolute ethanol (2×5 mL) and dried under reduced pressure to give crystalline zafirlukast ethanolate.

### Example 5

Preparation of Crystalline Zafirlukast Ethanolate by Re-Slurry of Zafirlukast Form X, Prepared According to U.S. Pat. No. 5,993,859, in Absolute Ethanol.

A mixture of zafirlukast (5.0 g) Form X, prepared according to U.S. Pat. No. 5,993,859, and absolute ethanol (50 mL) was stirred for 4 hours under reflux conditions, kept overnight at the room temperature and for 2 hours at 0-5° C. The precipitated crystals were filtered off, washed on filter with ice-cold absolute ethanol (2×5 mL) and dried under reduced pressure to give crystalline zafirlukast ethanolate.

### Example 6

Preparation of Crystalline Zafirlukast Ethanolate by Re-Slurry of Zafirlukast Form B, Prepared According to U.S. Pat. No. 5,993,859, in Absolute Ethanol.

A mixture of zafirlukast (5.0 g) Form B, prepared according to U.S. Pat. No. 5,993,859, and absolute ethanol (50 mL) was stirred for 4 hours under reflux conditions, kept overnight at the room temperature and 2 hours at 0-5° C. The precipitated crystals were filtered off, washed on filter with ice-cold absolute ethanol (2×500 mL) and dried under reduced pressure to give crystalline zafirlukast ethanolate.

### Example 7

Preparation of Crystalline Zafirlukast Ethanolate by Re-Slurry of Zafirlukast Form A, Prepared According to U.S. Pat. No. 5,993,859, in Absolute Ethanol.

A mixture of zafirlukast (5.0 g), Form A prepared according to U.S. Pat. No. 5,993,859, and absolute ethanol (50 mL) was stirred for 4 hours under reflux conditions, kept overnight at the room temperature and for 2 hours at 0-5° C. The precipitated crystals were filtered off, washed on filter with ice-cold absolute ethanol (2×5 mL) and dried under reduced pressure to give crystalline zafirlukast ethanolate.

### Example 8

Preparation of Crystalline Zafirlukast Ethanolate by Granulation of Zafirlukast Form A, Prepared According to J. Med. Chem., v. 33, 1781 (1990), in Absolute Ethanol.

A mixture of zafirlukast (4.0 g), Form A prepared according to J. Med. Chem., v. 33, 1781 (1990), and absolute ethanol (1.0 mL) was stirred in rotary evaporator at room temperature for 2 days. At the end of two days, the material contained crystalline ethanolate zafirlukast.

Although certain presently preferred embodiments of the invention have been described herein, it will be apparent to those skilled in the art to which the invention pertains that variations and modifications of the described embodiments may be made without departing from the spirit and scope of the invention. Accordingly, it is intended that the invention be limited only to the extent required by the appended claims and the applicable rules of law.

1. Solid ethanolate solvate form of zafirlukast (zafirlukast ethanolate).

2. Crystalline zafirlukast ethanolate.

3. Crystalline zafirlukast ethanolate according to claim 2, that exhibits an x-ray powder diffraction pattern having characteristic peaks expressed in degrees 20 at approximately 10.3, 12.7, 15.4, 18.3,20.1, 21.9 and 22.4.

4. A crystalline zafirlukast ethanolate according to claim 2, that exhibits a single crystal x-ray crystallographic analysis having
(a) crystal parameters that are approximately equal to the following:

| Formula | C₃₁H₃₃N₃O₆S.C₂H₅OH |
|---|---|
| Formula weight, amu | 621.73 |
| Crystal size (mm) | 0.4 × 0.3 × 0.15 |
| Space group | Cc |
| Crystal system | monoclinic |
| Temperature, K | 293(2) |
| Cell dimensions | a = 17.2160(10) Å |
| (Å = Angstrom | b = 11.0330(10) Å |
| ° = degree) | c = 18.2580(10) Å |
| | α = 90.00° |
| | β = 109.467(10)° |
| | γ = 90.00° |
| V (Å³) | 3269.7 |
| Z (molecules/unit cell) | 4 |
| density (g/cm³) | 1.263 |

wherein a, b and c and α, β and γ are as defined in the specification; and (b) the atomic positions of all atoms relative to the origin of the unit cell are as recited in Tables 2 and 6.

5. A crystalline zafirlukast ethanolate according to claim 2, having an infrared absorption spectrum in potassium bromide substantially as depicted in FIG. 4.

6. A method of preparing zafirlukast ethanolate comprising the steps of:
a) dissolving zafirlukast in solvent A;
b) adding absolute or aqueous ethanol to the zafirlukast solution obtained in step a);
c) optionally, removing partially or completely solvent A and excess of ethanol so that crystallization of the zafirlukast ethanolate begins and a suspension or slurry is formed; and
d) isolating the crystals of zafirlukast ethanolate.

7. A method of preparing zafirlukast ethanolate, comprising the steps of:
a) mixing zafirlukast with absolute or aqueous ethanol;
b) stirring the obtained mixture for a time sufficient to induce the transformation of zafirlukast to zafirlukast ethanolate; and
c) isolating the crystals of zafirlukast ethanolate.

8. The method of claim 6, wherein zafirlukast ethanolate is prepared by rapidly evaporating of a solution of zafirlukast in solvent A and absolute or aqueous ethanol at a temperature below the melting point of zafirlukast ethanolate.

9. The method of claim 6, wherein the rapid evaporation is carried out by spray drying.

10. The method of claim 6 or 7, further comprising the step of drying the crystals of zafirlukast ethanolate obtained in step d), at a temperature from about 30 to about 120° C.

11. The method of claim 6, further comprising the step of maintaining the suspension or slurry at a temperature from about −5 to about 80° C. for about 1 to about 18 hours.

12. The method of claim 6 or 8, wherein said solvent A is an organic solvent having similar or higher solubility of zafirlukast, as compared to absolute ethanol.

13. The method of claim 12, wherein said solvent A is selected from the groups consisting of chloroorganic solvents, ketones and nitrites.

14. The method of claim 13, wherein said solvent A is selected from dichloromethane, chloroform, acetone, methyl ethyl ketone (MEK) and acetonitrile.

15. The method of claim 6, wherein the suspension or slurry is cooled to below 20° C. prior to isolating the crystals of zafirlukast ethanolate.

16. The method of claim 6 or 7, wherein crystalline zafirlukast ethanolate exhibits an x-ray powder diffraction pattern having characteristic peaks expressed in degrees 2θ at approximately 10.3, 12.7, 15.4, 18.3, 20.1, 21.9 and 22.4.

17. The method of claim 6 or 7, wherein the crystalline zafirlukast ethanolate exhibits a single crystal x-ray crystallographic analysis having
(a) crystal parameters that are approximately-equal to the following:

| Formula | C₃₁H₃₃N₃O₆S.C₂H₅OH |
|---|---|
| Formula weight, amu | 621.73 |
| Crystal size (mm) | 0.4 × 0.3 × 0.15 |
| Space group | Cc |
| Crystal system | monoclinic |
| Temperature, K | 293(2) |
| Cell dimensions | a = 17.2160(10) Å |
| (Å = Angstrom | b = 11.0330(10) Å |
| ° = degree) | c = 18.2580(10) Å |
| | α = 90.00° |
| | β = 109.467(10)° |
| | γ = 90.00° |
| V (Å³) | 3269.7 |
| Z (molecules/unit cell) | 4 |
| density (g/cm³) | 1.263 |

wherein a, b and c and α, β and γ are as defined in the specification; and (b) atomic positions of all atoms relative to the origin of the unit cell as recited in Tables 2 and 6.

18. The method of claim 6 or 7, wherein zafirlukast ethanolate is characterized by an infrared spectrum pattern substantially as depicted in FIG. 4.

19. A pharmaceutical composition comprising a therapeutically effective amount of the compound of claim 1 or 2 and pharmaceutically acceptable carriers.

20. Use of crystalline zafirlukast ethanolate according to claim 1 or 2, in the manufacture of a medicament.

21. Method for the treatment of a disease state in which leukotrienes are implicated, comprising administering to a subject in need of such treatment, a therapeutically effective amount of a compound according to claim 1 or 2.
